(19) United States
(12) Patent Application Publication
(10) Pub. No.: US 2007/0129385 A1
(43) Pub. Date: Jun. 7, 2007

(54) Amorphous valganciclovir hydrochloride

(76) Inventors: Mukesh Kumar Sharma, Haryana (IN); Yatendra Kumar, Haryana (IN); Chandra Has Khanduri, Haryana (IN)

Correspondence Address: RANBAXY INC., 600 COLLEGE ROAD EAST, SUITE 2100, PRINCETON, NJ, 08540, US

(21) Appl. No.: 10/569,615

(22) PCT Filed: Aug. 27, 2004
(86) PCT No.: PCT/IB04/02789

§ 371 Date: Dec. 11, 2006

(30) Foreign Application Priority Data
Aug. 28, 2003 (IN) 1052/DEL/2003

(51) Int. Cl. A61K 31/522; C07D 473/10

(52) U.S. Cl. 514/263.38; 544/276

(57) ABSTRACT

The present invention relates to an amorphous form of valganciclovir hydrochloride and the pharmaceutical compositions thereof. The amorphous form can be directly prepared by spray-drying or azeotropic distillation of reaction mass. The amorphous form is useful in treating viral infections, for example, herpes simplex and cytomegalovirus.

### FIELD OF THE INVENTION

This invention relates to an amorphous form of valganciclovir hydrochloride; and processes for its preparation.

### BACKGROUND OF THE INVENTION

The L-valinate ester of 2-(2-amino-1,6-dihydro-6-oxo-purin-9-yl)-methoxy-3-hydroxy-1-propanyl hydrochloride salt commonly known as Valganciclovir hydrochloride of Formula I, is the mono-L-valyl ester prodrug of the antiviral compound ganciclovir.

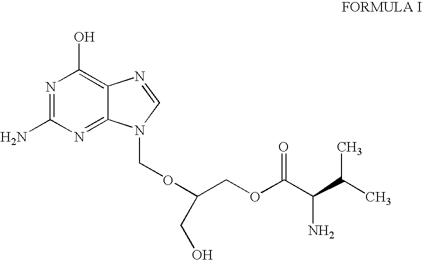

Ganciclovir is disclosed in U.S. Pat. No. 4,355,032. Ganciclovir inhibits replication of human cytomegalovirus in vitro and in vivo, and is effective against viruses of the herpes family, for example, against herpes simplex and cytomegalovirus. Ganciclovir is mostly used as an intravenous infusion, as it has a very low rate of absorption when administered orally.

Various mono and diacyl esters of ganciclovir are disclosed in J. Pharm. Sci. 76 (2), p. 180-184 (1987). The preparation of these esters is also mentioned in this article. However, the L-valyl ester of ganciclovir and its process of preparation are not discussed in this article.

European Patent No. 375329 discloses ester prodrugs of ganciclovir and physiologically acceptable salts thereof having advantageous bioavailability when administered by an oral route. The patent however, does not disclose the utility as well as process for the preparation of mono esters of ganciclovir.

U.S. Pat. Nos. 5,856,481; 5,840,890; 6,103,901; 5,700,936; 5,756,736 and 6,040,446 describe processes for the preparation and pharmaceutical compositions of mono L-valyl ester of ganciclovir (valganciclovir). The process provides various protecting groups and a methodology to prepare selectively mono L-valyl ester of ganciclovir. These processes provide crystalline valganciclovir hydrochloride.

U.S. Pat. No. 6,083,953 provides a process for preparing crystalline valganciclovir hydrochloride.

### SUMMARY OF THE INVENTION

It has been disclosed that amorphous forms of a number of drugs exhibit different dissolution characteristics and in some cases different bioavailability patterns compared to the corresponding crystalline forms [Konne T., Chem. Pharm. Bull., 38, 2003 (1990)]. For some therapeutic indications one bioavailability pattern may be favoured over another.

Amorphous valganciclovir hydrochloride is stable when stored under controlled humidity conditions and can be formulated into a suitable dosage form without conversion to a crystalline form. Solid amorphous valganciclovir hydrochloride is provided herein. The amorphous valganciclovir hydrochloride can be produced with no detectable crystalline valganchloride hydrochloride present, based on XRD investigations having a limit of detection of 0.5%. The amorphous form was found to have a superior stability profile to the existing crystalline form.

### BRIEF DESCRIPTION OF THE DRAWINGS

FIG. 1 is an X-ray powder diffraction (XRD) pattern of amorphous form of valganciclovir hydrochloride.

FIG. 2 is an X-ray powder diffraction (XRD) pattern of crystalline form of valganciclovir hydrochloride.

FIG. 3 is an X-ray powder diffraction (XRD) pattern of largely amorphous valganciclovir hydrochloride mixed with some crystalline form of valganciclovir hydrochloride.

### DETAILED DESCRIPTION OF THE INVENTION

In one aspect, herein is provided an amorphous form of valganciclovir hydrochloride of Formula I.

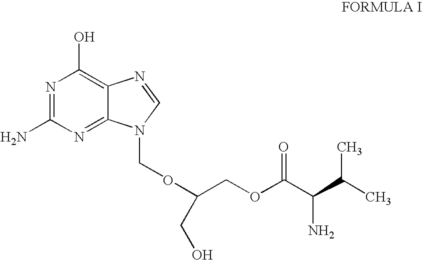

In another aspect, provides a process for preparing an amorphous form of valganciclovir hydrochloride wherein the process comprises
a) converting ganciclovir of Formula II, to the N-benzyloxycarbonyl-L-valinate ester of ganciclovir of Formula III,
b) removing the N-benzyloxycarbonyl group by hydrogenolysis,
c) converting valganciclovir to its hydrochloride salt,
d) concentrating the solution of hydrochloride salt to remove solvent, and
e) isolating an amorphous form of valganciclovir hydrochloride from the reaction mass thereof

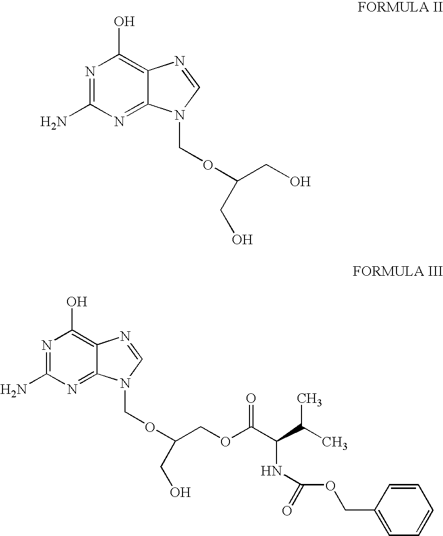

Ganciclovir of Formula II is first converted to N-benzyloxycarbonyl-L-valinate ester of ganciclovir of Formula III, as per a process described in U.S. Pat. No. 5,756,736. The protected mono valine ester of ganciclovir of Formula III is then deprotected by hydrogenolysis of N-benzyloxycarbonyl group in presence of palladium on carbon catalyst. The deprotection reaction is carried out in presence of an organic solvent such as ethanol, methanol, tetrahydrofuran, isopropanol and the like. The reaction can be carried out in presence of hydrogen gas in a pressurized vessel at temperatures of about 20 to about 100° C. A compound capable of generating hydrogen may also be used in the reaction wherein use of hydrogen gas can be avoided. Formic acid, sodium formate, ammonium formate, sodium acetate and acetic acid are examples of compounds which are capable of generating hydrogen gas. When formic acid is used as source of hydrogen, the reaction can be carried out at temperatures of about 25 to about 50° C. After completion of the reaction the catalyst is filtered and the filtrate concentrated under vacuum. The residue, after treating with hydrochloric acid, is dissolved in an organic solvent and from the solution solvent is removed to get amorphous valganciclovir hydrochloride.

The organic solvent can be selected from, for example lower alkanols, esters, ethers, aromatic hydrocarbons, chlorinated hydrocarbons, ketones, acetonitrile, polar protic and polar aprotic solvents or mixtures thereof. Lower alkanols can be selected from C₁₋₅ straight or branched chain alcohols such as methanol, ethanol, n-propanol, isopropanol, n-butanol, iso-butanol, sec-butanol and tert-butanol. Esters can be selected from C₁₋₆ straight or branched chain ester such as methyl formate, ethyl formate, methyl acetate, ethyl acetate, n-propyl acetate, isopropyl acetate, n-butyl acetate and isobutyl acetate. Ethers can be selected from C₁₋₆ straight or branched chain or C₁₋₆ cyclic ethers such as diethyl ether, diisopropyl ether, tetrahydrofuran and 1,4-dioxane. Aromatic hydrocarbons can be selected from benzene, toluene, and xylene, substituted toluenes and substituted xylenes. Chlorinated hydrocarbons can be selected from methylene chloride, ethylene chloride, chloroform, methylene bromide, ethylene bromide and carbon tetrachloride. Ketones can be selected from C₁₋₇ straight or branched chain or cyclic ketones selected from acetone, ethyl methyl ketones, diisobutyl ketone and methyl isobutyl ketone. Polar protic and polar aprotic solvent can be selected from N,N-dimethylformamide, N,N-dimethylacetamide and dimethylsulphoxide.

The solvent from the solution of valganciclovir hydrochloride can be removed by a spray-drying technique. For the purpose of spray-drying, mini-spray Dryer (Model: Buchi 190 Switzerland) which operates on the principle of nozzle spraying in an parallel-flow, i.e., the sprayed product and the drying gas flow in the same direction was used. The drying gas can be air or inert gases such as nitrogen, argon or carbon dioxide. Nitrogen is used in particular embodiments.

Alternatively, the solution of valganciclovir hydrochloride obtained can be concentrated under vacuum to remove the solvent, thus obtaining an amorphous form of valganciclovir hydrochloride. The so-obtained amorphous product can then be dried under vacuum.

In yet another aspect, herein is provided a process of making amorphous valganciclovir hydrochloride wherein the process comprises
a) dissolving crystalline valganciclovir hydrochloride an aqueous solvent, such as water, optionally containing a suitable organic solvent,
b) removing solvent from the solution obtained in step a), and
c) isolating amorphous valganciclovir hydrochloride.

Crystalline valganciclovir hydrochloride was prepared by process as reported in U.S. Pat. No. 6,083,953. The crystalline material is then dissolved in water, optionally containing an organic solvent. From the solution, the solvent is then removed either by vacuum distillation or by a spray-drying technique to yield an amorphous form of valganciclovir hydrochloride.

The organic solvent used for dissolving valganciclovir hydrochloride can be selected from a group of water-miscible organic solvents such as C₁₋₄ straight or branched chain lower alkanols such as methanol, ethanol, n-propanol and isopropanol; acetone, acetonitrile, tetrahydrofuran, 1,4-dioxane or mixtures thereof.

In yet a further aspect, herein is provided processes for the preparation of amorphous valganciclovir hydrochloride wherein the process comprises
a) dissolving crystalline valganciclovir hydrochloride in water,
b) adding an organic solvent capable of forming an azeotropic mixture with water,
c) azeotropically removing water from the mixture of step b),
d) treating the mixture obtained in step c) with a further organic solvent, and
e) isolating amorphous valganciclovir hydrochloride from the reaction mass thereof.

Crystalline valganciclovir hydrochloride was prepared by processes reported in U.S. Pat. No. 6,083,953. The crystalline material was dissolved in water and to the solution was added an organic solvent capable of forming an azeotropic mixture with water. The resultant mass is then concentrated to completely remove water azeotropically. The organic solvent capable of removing water azeotropically can be selected from for example, ethanol, isopropanol, n-butanol, methylene chloride, chloroform, carbon tetrachloride, toluene, xylene, ethyl acetate, methyl acetate, tetrahydrofuran, acetone or mixtures thereof. After substantially complete removal of water, the organic solvent is also removed under vacuum. The residue obtained is treated with a further organic solvent for sufficient time to substantially precipitate the product which is then filtered and dried under vacuum to afford amorphous valganciclovir hydrochloride. The second organic solvent can be selected from for example, acetone, isopropanol, tetrahydrofuran, cyclohexane, n-hexane, ethyl acetate, diethyl ether and diisopropyl ether.

In a still further aspect, herein is provided a process for converting a mixture of amorphous and crystalline form of valganciclovir hydrochloride to substantially amorphous valganciclovir.

A mixture of amorphous valganciclovir hydrochloride with some crystalline valganciclovir hydrochloride can be prepared directly from the reaction mixture. For example, the preparation can comprise from about 5% to about 20% by weight of crystalline valganciclovir hydrochloride based on total sample weight. The nature of the solvent used for precipitation is believed to affect the extent of crystallinity in valganciclovir hydrochloride formed. For example, when acetone is used as precipitant, crystalline material tends to be formed, whereas with use of isopropanol, crystalline material was not detected. The rate of addition of a second organic solvent also appears to play a role, with foster addition resulting in less crystalline, and slower resulting in more.

After completion of the reaction, the catalyst can be removed by filtration and the filtrate concentrated under vacuum. An organic solvent capable of removing water azeotropically is added to the residue. The organic solvent can be selected from, for example, ethanol, isopropanol, n-butanol, methylene chloride, chloroform, carbon tetrachloride, toluene, xylene, ethyl acetate, methyl acetate, tetrahydrofuran, acetone or mixtures thereof. After removal of water, the organic solvent is also removed under vacuum. Desirably, water is completely removed at this stage to minimize the formation of crystalline material. For example, the presence of about 5% water in the reaction mass gives about 5-8% crystalline material. The residue obtained is treated with another organic solvent for a time sufficient to precipitate the product which is filtered and dried under vacuum to get a mixture containing mostly an amorphous form, with some crystalline form of valganciclovir hydrochloride. The organic solvent can be selected from for example, acetone, isopropanol, tetrahydrofuran, cyclohexane, n-hexane, ethyl acetate, diethyl ether and diisopropyl ether.

The mixture of amorphous valganciclovir hydrochloride containing some crystalline form is then substantially converted to an amorphous form by dissolving it in water (optionally containing an organic solvent) followed by spray-drying of the solution as described earlier. Crystalline formation at this stage can be inhibited by storage under nitrogen in the strict absence of atmosphere or other water. The amorphous material has been found stable for up to at least 3 months with proper storage.

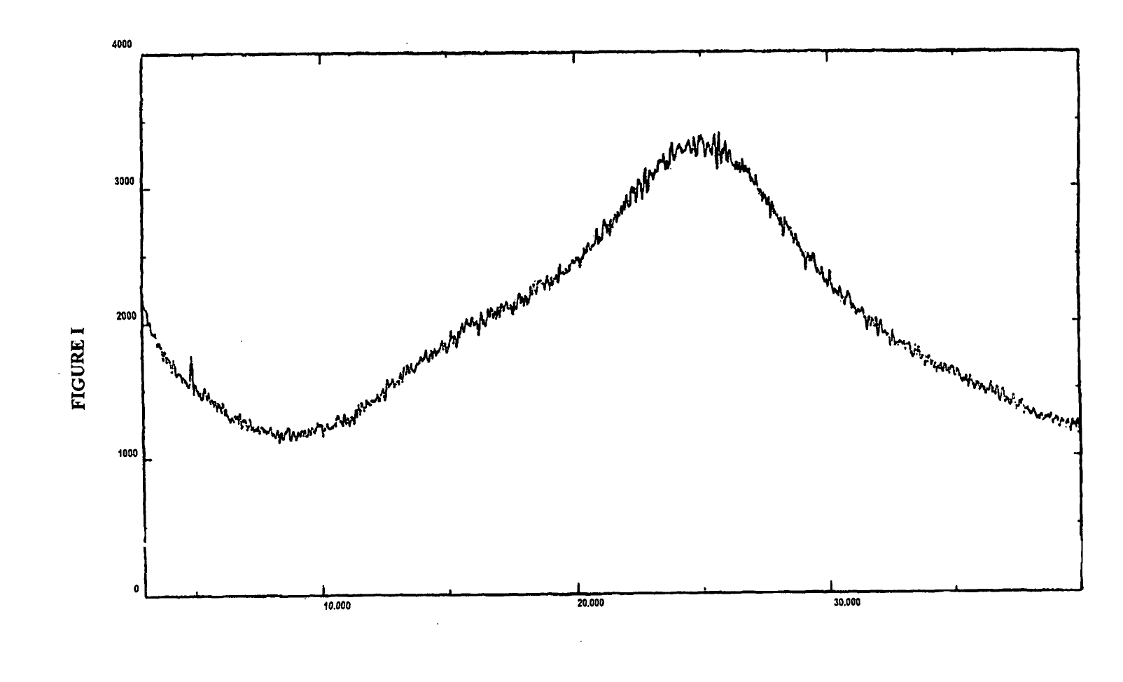

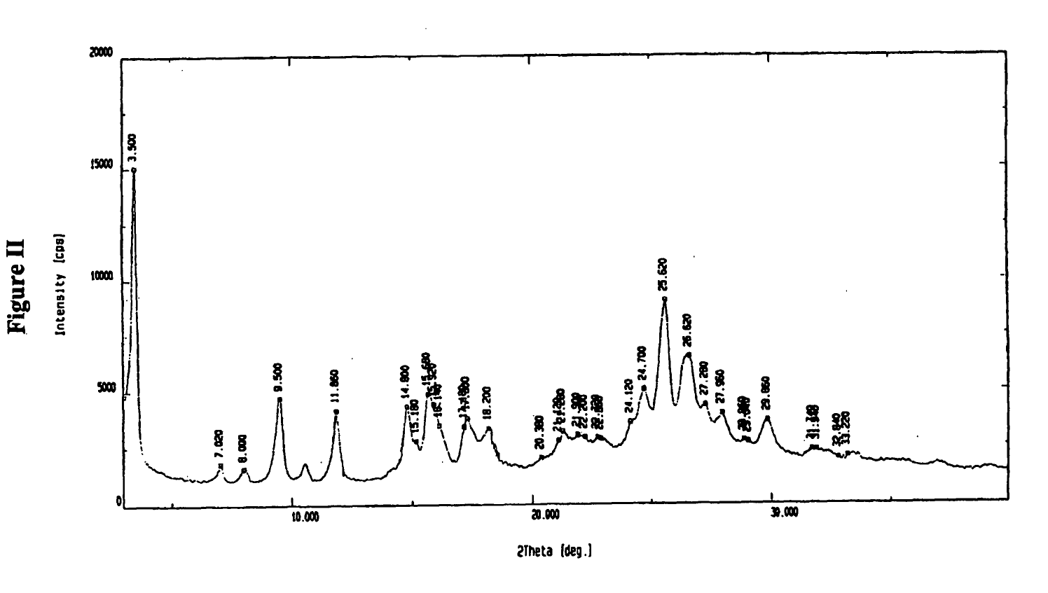

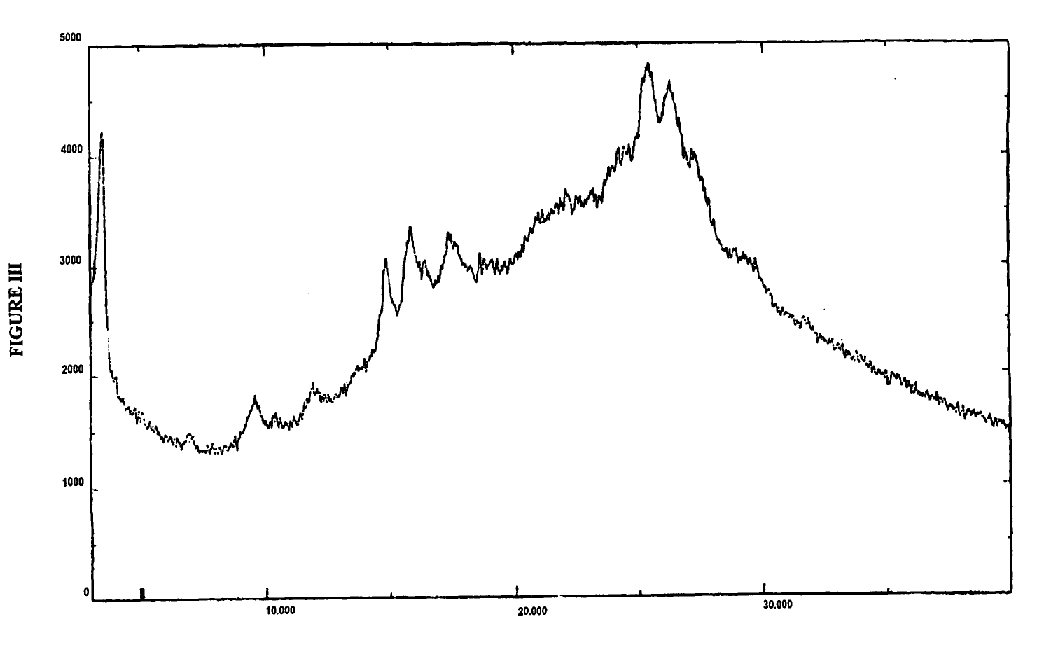

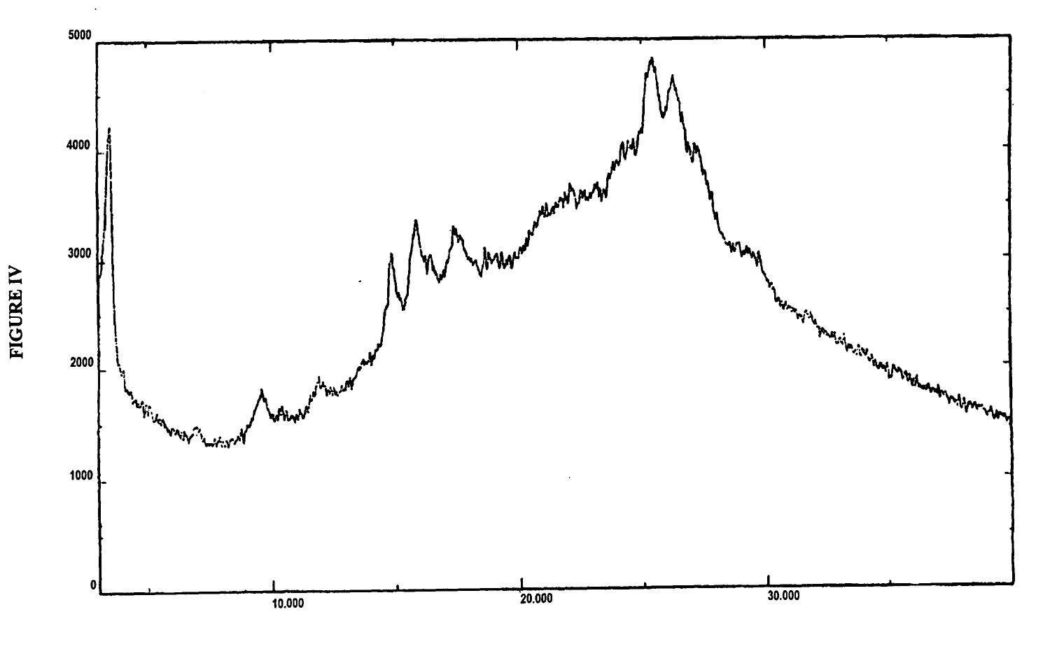

In a further aspect, herein is provided amorphous valganciclovir hydrochloride having a XRD pattern, as shown in FIG. 1, which is different than the XRD pattern of crystalline valganciclovir hydrochloride, as shown in FIG. 2.

In yet another aspect, herein is provided a pharmaceutical composition comprising amorphous valganciclovir hydrochloride along with pharmaceutically acceptable carriers and/or diluents.

The compositions include dosage forms suitable for oral, buccal, rectal, and parenteral (including subcutaneous, intramuscular, and ophthalmic administration). Dosage forms include solid dosage forms, like powders, tablets (which can be conventional, sustained release or controlled release), capsules, suppositories, sachets, troches and lozenges as well as liquid suspensions, emulsions, pastes and elixirs. Parenteral dosage forms can include intravenous infusions, sterile solutions for intramuscular, subcutaneous or intravenous administration, dry powders to be reconstituted with sterile water for parenteral administration and the like.

In still another aspect, herein is provided to a method of treating viral infections such as herpes simplex and cytomegalovirus, comprising administering to a mammal in need thereof therapeutically effective amount of amorphous valganciclovir hydrochloride.

While the present invention has been described in terms of its specific embodiments, certain modifications and equivalents will be apparent to those skilled in the art and are included within the scope of the claims.

### EXAMPLES

### Example 1

Preparation of Amorphous Form of Valganciclovir from Reaction Mixture by Spray-Drying.

Mono CBZ-L-valine ganciclovir of Formula III (45 g) in ethanol (585 ml) was heated to get a clear solution followed by cooling to 40° C., formic acid was added (85%, 10.57 g), as well as water (58.5 ml) and palladium on carbon catalyst (5%, 50% wet, 7.5 g). The reaction mixture was stirred at 40-45° C., for 3-4 hrs. After completion of the reaction, the catalyst was removed by filtration through celite bed which was then washed with ethanol (45 ml). The filtrate was recovered at 25-35° C. under vacuum, and to the residue was added water (112 ml) and concentrated hydrochloric acid (9.3 ml). The mixture was filtered to remove undissolved material and the cake was washed with water (22.5 ml). To the clear filtrate was added IPA (96 ml) and the resultant mixture was warmed to 40° C. to get a clear solution. The clear solution was spray dried at 70-75° C., 6.0 kg nitrogen pressure and at a rate of about 1.5 ml per minute.

The material was recovered from receiver and dried at 40-45° C. under vacuum for 6 hrs.

Yield—16 g (XRD as per FIG. 1 showed it to be an amorphous material). No crystalline material was detected.

### Example 2

Conversions of Crystalline Form into Amorphous by Spray Drying.

Step a) Preparation of Crystalline Valganciclovir Hydrochloride

Mono CBZ-L-valine ganciclovir of Formula III (40 g) in ethanol (commercial, 500 ml) was heated to get a clear solution, followed by cooling to 40° C., formic acid was added (85%, 12.26 g), as well as water (50 ml) and palladium on carbon catalyst (5%, 50% wet, 8.0 g). The reaction mixture was stirred at 40-45° C., for 3-4 hrs. After completion of the reaction, the catalyst was removed by filtration through celite bed which was then washed with ethanol (20 ml). Added concentrated hydrochloric acid (8.3 ml) and the filtrate was concentrated completely at 25-35° C. under vacuum, and to the residue added ethanol absolute (100 ml) and recovered completely at 25-35° C. to remove water. To the concentrated mass added absolute ethanol (160 ml) and stirred at 25-30° C. for 1 hr. Filtered the solid and washed with absolute ethanol (30 ml). The product was dried at 40-45° C. under vacuum for 12 hrs.

Yield—24.2 g (XRD as per FIG. 2 showed it to be a crystalline form of valganciclovir hydrochloride).

Step b) Conversion of Crystalline Valganciclovir Hydrochloride to Amorphous Valganciclovir Hydrochloride

The material obtained in step a) (10 g) was dissolved in water (35 ml) filtered to remove any undissolved particle and washed with water (5 ml). The clear solution was spray dried at 70-75° C., 6.0 kg nitrogen pressure and at a rate of about 1.5 ml per minute. The material was recovered from receiver and dried at 40-45° C. under vacuum for 6 hrs.

Yield—8.0 g (XRD as per FIG. 1 showed it to be an amorphous material). No crystalline material was detected.

### Example 3

Preparation of Mixture of Amorphous and Crystalline and its Conversion to Amorphous Form

Step a) Preparation of Mixture of Amorphous and Crystalline Valganciclovir Hydrochloride

Mono CBZ-L-valine ganciclovir of Formula III (45 g) in ethanol (commercial, 585 ml) was heated get a clear solution, followed by cooling to 40° C., formic acid was added (85%, 13.8 g), as well as water (58.5 ml) and palladium on carbon catalyst (5%, 50% wet, 10.12 g). The reaction mixture was stirred at 40-45° C., for 3-4 hrs. After completion of the reaction, the catalyst was removed by filtration through celite bed which was then washed with ethanol (20 ml). Concentrated hydrochloric acid was added (9.3 ml) and the filtrate was concentrated completely at 25-35° C. under vacuum, absolute ethanol was added (100 ml) and recovered at 25-35° C. to remove water. To the concentrated mass was added acetone (315 ml) and stirred at 25-30° C. for 12 hrs. The solid was filtered and washed with acetone (90 ml). The product was dried at 40-45° C. under vacuum for 12 hrs.

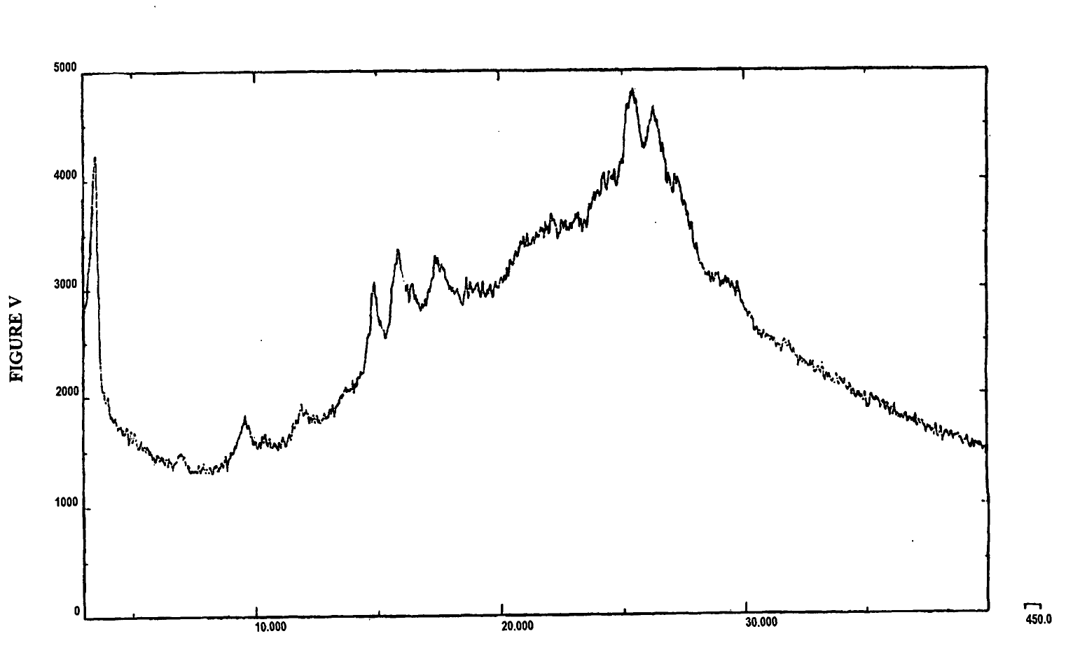

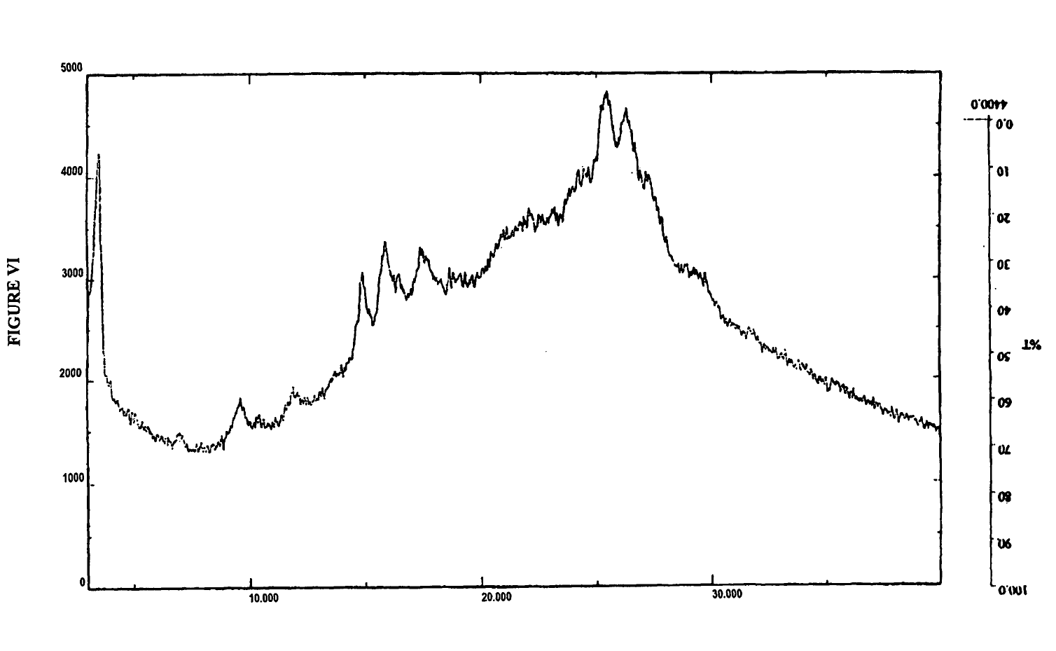

Yield—35 g (XRD of this material as per FIG. 3 showed it to be a mixture of largely amorphous material mixed with some crystalline material, approximately 15% by weight)

Step b) Conversion of Mixture of Amorphous and Crystalline Valganciclovir Hydrochloride to Amorphous Valganciclovir

The product obtained from step a) (10 g) of example 3 was dissolved in water (35 ml) filtered to remove undissolved particles and washed with water (5 ml). The clear solution was spray dried at 70-75° C., 6.0 kg nitrogen pressure and at a rate of about 1.5 ml per minute. The material was recovered from receiver and dried at 40-45° C. under vacuum for 6 hrs.

Yield—8.0 g (XRD as per FIG. 1 showed it to be an amorphous material). No crystalline material was detected.

### Example 4

Conversion of Mixture of Amorphous and Crystalline Valganciclovir Hydrochloride to Amorphous Valganciclovir

The product obtained from step a) (5 g) of Example 3 was dissolved in water (50 ml), filtered to remove undissolved material and washed with water (5 ml). The clear solution was spray dried at 70-75° C., 6.0 kg nitrogen pressure and at a rate of about 1.5 ml per minute. The material was recovered from the receiver and dried at 40-45° C. under vacuum for 6 hrs.

Yield—3.5 g (XRD as per FIG. 1 showed it to be an amorphous material). No crystalline material was detected.

### Example 5

Conversion of Mixture of Amorphous and Crystalline Valganciclovir Hydrochloride to Amorphous Valganciclovir

The product obtained from step a) (5 g) of Example 3 was dissolved in water (20 ml), filtered to remove undissolved material and washed with water (5 ml). To the clear filtrate added isopropanol (20 ml). The clear solution was spray dried at 70-75° C., 6.0 kg nitrogen pressure and at a rate of about 1.5 ml per minute. The material was recovered from receiver and dried at 40-45° C. under vacuum for 6 hrs.

Yield—3.5 g (XRD as per FIG. 1 showed it to be an amorphous material). No crystalline material was detected.

### Example 6

Preparation of Amorphous Form of Valganciclovir Hydrochloride from Reaction Mass by Precipitation from IPA

Mono CBZ-L-valine ganciclovir (10 g) in ethanol (130 ml) was heated to get a clear solution, followed by cooling to 40° C., formic acid (85%, 2.35 ml) and palladium on carbon catalyst were added (10%, 50% wet, 2.0 g). The reaction mixture was stirred at 40-45° C., for 3-4 hrs. After completion of the reaction, the catalyst was removed by filtration through celite bed which was then washed with ethanol (45 ml). The filtrate was recovered completely at 25-35° C. under vacuum, and to the residue was added water (25 ml) and concentrated hydrochloric acid (2.0 ml). The mixture was washed twice with ethyl acetate (25 ml) and to the aqueous layer added isopropanol (30 ml). Solvent was recovered at 25-35° C. under vacuum, to the residue to which was added isopropanol (30 ml) and recovered solvent. The water present in the reaction mass was once again removed azeotropically using isopropanol to ensure substantially complete water removal. Isopropanol was added (30 ml) and stirred overnight. The solid was filtered and washed with 10 ml IPA. The product was dried at 40-45° C. under vacuum for 12 hrs.

Yield—4.0 g (XRD as per FIG. 1 showed it to be an amorphous material). No crystalline material was detected.

We claim:

1. Amorphous valganciclovir hydrochloride.

2. Amorphous valganciclovir hydrochloride having a purity of at least about 98%.

3. Amorphous valganciclovir hydrochloride having less than 20% of crystalline valganciclovir hydrochloride.

4. A pharmaceutical composition comprising amorphous valganciclovir hdrochloride and pharmaceutically acceptable carriers and/or diluents.

5. A method of treating viral infections, comprising administering to a mammal in need thereof a therapeutically effective amount of amorphous valganciclovir hydrochloride.

6. The method of claim 4, wherein the virus is selected from herpes simplex and cytomegalovirus.

7. A process for preparing an amorphous form of valganciclovir hydrochloride wherein the process comprises
a) converting ganciclovir of Formula II to the N-benzyloxycarbonyl-L-valinate ester of ganciclovir of Formula III,
b) removing the N-benzyloxycarbonyl group by hydrogenolysis,
c) converting valganciclovir to its hydrochloride salt in presence of an aqueous solvent,
d) concentrating the solution of hydrochloride salt to remove solvent, and
e) isolating amorphous valganciclovir hydrochloride.

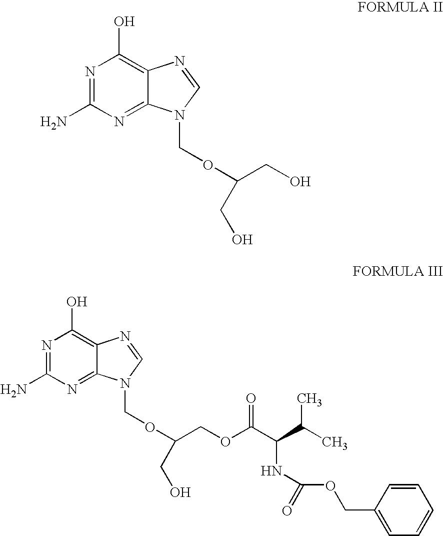

8. A process of making amorphous valganciclovir hydrochloride wherein the process comprises
a) dissolving crystalline valganciclovir hydrochloride in an aqueous solvent,
b) removing solvent from the solution obtained in step a), and
c) isolating amorphous valganciclovir hydrochloride.

9. The process of claim 6 wherein the hydrogenolysis is carried out using palladium on carbon.

10. The process of claim 6 wherein the hydrogenolysis is carried out using hydrogen gas or compound capable of generating hydrogen.

11. The process of claim 9 wherein the compound capable of producing hydrogen gas is selected from formic acid, sodium formate, ammonium formate, sodium acetate and acetic acid.

12. The process of claim 6 wherein an aqueous solution of hydrochloric acid, concentrated hydrochloric acid or hydrogen chloride gas is used for formation of the hydrochloride salt.

13. The process of claim 6 or 7 wherein the organic solvent is selected from C₁₋₅ straight or branched chain lower alkanols, C₁₋₆ straight or branched chain ester, C₁₋₆ straight or branched chain or C₁₋₆ cyclic ethers, aromatic hydrocarbons, chlorinated hydrocarbons, C₁₋₇ straight or branched chain or cyclic ketones, acetonitrile, polar protic and polar aprotic solvents or mixtures thereof.

14. The process of claim 12 wherein the lower alkanol is methanol, ethanol, n-propanol, isopropanol, n-butanol, iso-butanol, sec-butanol or tert-butanol.

15. The process of claim 12 wherein the ester is methyl formate, ethyl formate, methyl acetate, ethyl acetate, n-propyl acetate, isopropyl acetate, n-butyl acetate or isobutyl acetate.

16. The process of claim 12 wherein the ether is diethyl ether, diisopropyl ether, tetrahydrofuran or 1,4-dioxane.

17. The process of claim 12 wherein the aromatic hydrocarbon is benzene, toluene, and xylene, substituted toluenes or substituted xylenes.

18. The process of claim 12 wherein the chlorinated hydrocarbon is methylene chloride, ethylene chloride, chloroform, methylene bromide, ethylene bromide or carbon tetrachloride.

19. The process of claim 12 wherein the ketone is acetone, ethyl methyl ketones, diisobutyl ketone or methyl isobutyl ketone.

20. The process of claim 12 wherein the polar protic and polar aprotic solvent is N-methylpyrrolidone, N,N-dimethylformamide, N,N-dimethylacetamide or dimethylsulphoxide.

21. The process of claim 6 or 7 wherein concentration for removal of solvent is carried out by spray-drying.

22. The process of claim 6 or 7 wherein concentration for removal of solvent is carried out by distillation under vacuum.

23. The process of claim 6 or 7 wherein isolated amorphous valganciclovir hydrochloride.

24. A process for the preparation of amorphous valganciclovir hydrochloride wherein the process comprises
a) dissolving crystalline valganciclovir hydrochloride in water,
b) adding an organic solvent capable of forming an azeotropic mixture with water,
c) azeotropically removing water from the mixture of step b)
d) treating the mixture obtained in step c) with a further organic solvent, and
e) isolating amorphous valganciclovir hydrochloride.

25. The process of claim 23 wherein the organic solvent capable of forming an azeotropic mixture is ethanol, isopropanol, n-butanol, methylene chloride, chloroform, carbon tetrachloride, toluene, xylene, ethyl acetate, methyl acetate, tetrahydrofuran, acetone or mixtures thereof.

26. The process of claim 23 wherein after azeotropic removal of water the organic solvent is also removed.

27. The process of claim 23 wherein the further organic solvent is acetone, isopropanol, tetrahydrofuran, cyclohexane, n-hexane, ethyl acetate, diethyl ether or diisopropyl ether.

28. The process of claim 23 wherein isolated amorphous valganciclovir hydrochloride is dried under vacuum.

29. A process for preparation of amorphous valganciclovir hydrochloride from a mixture of amorphous and crystalline valganciclovir hydrochloride by spray-drying or vacuum distillation technique.
